(19) United States
(12) Patent Application Publication
(10) Pub. No.: US 2008/0267972 A1
(43) Pub. Date: Oct. 30, 2008

(54) Donor Lymphocyte Infusion of T Cells For the Treatment of Cancer

(75) Inventor: Ronald J. Berenson, Mercer Island, WA (US)

Correspondence Address: INVITROGEN CORPORATION;C/O INTELLEVATE, P.O. BOX 52050, MINNEAPOLIS, MN, 55402, US

(73) Assignee: Invitrogen Corporation, Carlsbad, CA (US)

(21) Appl. No.: 11/815,964

(22) PCT Filed: Feb. 10, 2006
(86) PCT No.: PCT/US2006/004895

§ 371 Date: Jun. 13, 2008

Related U.S. Application Data
(60) Provisional application No. 60/652,068, filed on Feb. 10, 2005.

(51) Int. Cl. A61K 35/14; A61K 39/395; A61P 35/00

(52) U.S. Cl. 424/152.1; 424/93.71; 424/172.1

(57) ABSTRACT

The present invention relates generally to the treatment of cancer using donor lymphocyte infusions of T cells. Further, the present invention relates generally to methods for stimulating and activating allogeneic and/or xenogeneic cells which can then be used in a DLI setting.

### TECHNICAL FIELD

The present invention relates generally to the treatment of cancer using T cells, in particular, donor lymphocyte infusions of T cells. Further, the present invention relates generally to methods for stimulating and activating cells which can then be used in a DLI setting.

### BACKGROUND OF THE INVENTION

Donor lymphocyte infusion (DLI) is a relative recent procedure developed in the late 1980s for the treatment of a variety of malignancies, most notably, in a number of hematopoietic malignancies, some solid tumor malignancies, in mediating viral modulation effects (e.g., preventing cytomegalovirus (CMV) symptoms resulting from immunosuppression), and as a prophylactic approach in preventing of recurrent disease. In particular, DLI is used after stem cell transplantation and is central to generating graft-versus-leukemia (GVL), a crucial component of cancer therapy. While the GVL effect is usually accompanied by undesirable graft versus host disease (GVHD), in some cases the GVL effect can be induced without any noticeable GVHD.

DLI is usually used in the setting of allogeneic stem cell transplant, following significant conditioning to enable the donor stem cells to engraft. Inflammatory stimuli, such as cytokines induced by myeloablative conditioning treatments may be required to activate endothelial cells and, in combination with activation-induced increased function of T-cell adhesion molecules, permit T-cell adhesion and migration into GVHD target tissues. Unfortunately, current methods for conditioning of patients for allogeneic BMT are particularly harsh and lead to activated inflammatory stimuli, thus explaining patients' exquisite sensitivity to the development of GVHD.

Thus, there is a need in the art for a reduction or elimination of the need for such harsh conditioning treatments while maintaining the desired GVL effect. The present invention fills this and other related needs.

### SUMMARY OF THE INVENTION

The present invention provides methods for treating cancer comprising administering allogeneic T cells to a cancer patient, wherein said patient has not received a conditioning regimen. In one embodiment, the T cells are activated T cells. In a further embodiment, T cells are activated by a method comprising, contacting a population of allogeneic cells from a suitable donor, wherein at least a portion of the population comprises T cells, with a surface, wherein said surface has attached thereto a first agent which stimulates a TCR/CD3 complex-associated signal in the T cells and a second agent that binds the CD28 accessory molecule on the surface of the T cells, thereby activating the T cells. In this regard, a suitable donor refers to a donor that has been suitably matched as described further herein. In another embodiment, the first agent is an antibody or an antigen-binding fragment thereof and in certain embodiments, is a monoclonal antibody or antigen-binding fragment thereof. In one embodiment, the antibody is an anti-CD3 antibody. In an additional embodiment, the second agent is an antibody or an antigen-binding fragment thereof and in certain embodiments, the antibody is a monoclonal antibody or antigen-binding fragment thereof. In one embodiment, the antibody is an anti-CD28 antibody.

In a further embodiment the first and the second agents are both antibodies or antigen-binding fragments thereof. In this regard, the first agent may be an anti-CD3 antibody or antigen-binding fragments thereof and the second agent may be an anti-CD28 antibody or antigen-binding fragments thereof. In certain embodiments, the second agent is a natural ligand of CD28, such as, B7-1. In another embodiment, the surface can be a solid surface, a cell surface, or a paramagnetic bead.

In further embodiments, the first and second agents are covalently or noncovalently attached to the surface. In a further embodiment, the first and second agents are indirectly attached to the surface.

In an additional embodiment, the cancers that can be treated with the methods and compositions described herein include, but are limited to NHL, CLL, and CML.

### DETAILED DESCRIPTION OF THE INVENTION

Prior to setting forth the invention, it may be helpful to an understanding thereof to set forth definitions of certain terms that will be used hereinafter.

The term “stimulation”, as used herein, refers to a primary response induced by ligation of a cell surface moiety. For example, in the context of receptors, such stimulation entails the ligation of a receptor and a subsequent signal transduction event. With respect to stimulation of a T cell, such stimulation refers to the ligation of a T cell surface moiety that in one embodiment subsequently induces a signal transduction event, such as binding the TCR/CD3 complex. Further, the stimulation event may activate a cell and upregulate or downregulate expression or secretion of a molecule, such as downregulation of TGF-β. Thus, ligation of cell surface moieties, even in the absence of a direct signal transduction event, may result in the reorganization of cytoskeletal structures, or in the coalescing of cell surface moieties, each of which could serve to enhance, modify, or alter subsequent cell responses.

The term “activation”, as used herein, refers to the state of a cell following sufficient cell surface moiety ligation to induce a noticeable biochemical or morphological change. Within the context of T cells, such activation, refers to the state of a T cell that has been sufficiently stimulated to induce cellular proliferation. Activation of a T cell may also induce cytokine production and performance of regulatory or cytolytic effector functions. Within the context of other cells, this term infers either up or down regulation of a particular physico-chemical process.

The term “force”, as used herein, refers to an artificial or external force applied to the cells to be stimulated that induces cellular concentration and concentration of cells with the agent that binds a cell surface moiety. For example, the term “force” includes any force greater than gravity (i.e., in addition to gravity and not solely gravitational force) that induces cell concentration and/or cell surface moiety aggregation. Such forces include transmembrane pressure such as filtration, a hydraulic force, an electrical force, an acoustical force, a centrifugal force, or a magnetic force. Ideally, the force utilized drives the concentration of the target cell of interest with an agent that ligates a cell surface moiety. In various contexts, the force can be pulsed, i.e., applied and reapplied (e.g., a magnetic force could be turned off and on, pulsing the population of cells in combination with a paramagnetic particle).

The term “simultaneous”, as used herein, refers to the fact that inherently upon concentrating cells at a surface that has cell surface moiety binding agents attached thereto, results in concentration of cells with each other and with the surface, thus ligands (i.e., agents). However, the use of the term “simultaneous” does not preclude previous binding of the target cells with a surface having cell surface moiety binding agents attached thereto, as concentration and further ligand binding occurs simultaneously at the concentration surface. For example, within the context of T cell activation, the T cells may be exposed to a surface such as a paramagnetic bead having anti-CD3 and anti-CD28 antibodies attached thereto and subsequently concentrated by a magnetic field. Thus, in this context while cells and beads have previous contact and ligation, nevertheless, during concentration of cells additional ligation occurs.

The term “target cell”, as used herein, refers to any cell that is intended to be stimulated by cell surface moiety ligation.

An “antibody”, as used herein, includes both polyclonal and monoclonal antibodies; primatized (e.g., humanized); murine; mouse-human; mouse-primate; and chimeric; and may be an intact molecule, a fragment thereof (such as scFv, Fv, Fd, Fab, Fab′ and F(ab)′₂ fragments), or multimers or aggregates of intact molecules and/or fragments; and may occur in nature or be produced, e.g., by immunization, synthesis or genetic engineering; an “antibody fragment,” as used herein, refers to fragments, derived from or related to an antibody, which bind antigen and which in some embodiments may be derivatized to exhibit structural features that facilitate clearance and uptake, e.g., by the incorporation of galactose residues. This includes, e.g., F(ab), F(ab)′₂, scFv, light chain variable region (V_{L}), heavy chain variable region (V_{H}), and combinations thereof.

The term “protein”, as used herein, includes proteins, polypeptides and peptides; and may be an intact molecule, a fragment thereof, or multimers or aggregates of intact molecules and/or fragments; and may occur in nature or be produced, e.g., by synthesis (including chemical and/or enzymatic) or genetic engineering.

The term “agent”, “ligand”, or “agent that binds a cell surface moiety”, as used herein, refers to a molecule that binds to a defined population of cells. The agent may bind any cell surface moiety, such as a receptor, an antigenic determinant, or other binding site present on the target cell population. The agent may be a protein, peptide, antibody and antibody fragments thereof, fusion proteins, synthetic molecule, an organic molecule (e.g., a small molecule), or the like. Within the specification and in the context of T cell stimulation, antibodies are used as a prototypical example of such an agent.

The terms “agent that binds a cell surface moiety” and “cell surface moiety”, as used herein, are used in the context of a ligand/anti-ligand pair. Accordingly, these molecules should be viewed as a complementary/anti-complementary set of molecules that demonstrate specific binding, generally of relatively high affinity (an affinity constant, Kₐ, of about 10⁶ M⁻¹).

A “co-stimulatory signal”, as used herein, refers to a signal, which in combination with a primary signal, such as TCR/CD3 ligation, leads to T cell proliferation.

A “ligand/anti-ligand pair”, as used herein, refers to a complementary/anti-complementary set of molecules that demonstrate specific binding, generally of relatively high affinity (an affinity constant, Kₐ, of about 10⁶ M⁻¹,). Exemplary ligand/anti-ligand pairs enzyme/inhibitor, hapten/antibody, lectin/carbohydrate, ligand/receptor, and biotin/avidin or streptavidin. Within the context of the present invention specification receptors and other cell surface moieties are anti-ligands, while agents (e.g., antibodies and antibody fragments) reactive therewith are considered ligands.

“Separation”, as used herein, includes any means of substantially purifying one component from another (e.g., by filtration or magnetic attraction).

“Quiescent”, as used herein, refers to a cell state wherein the cell is not actively proliferating.

A “surface”, as used herein, refers to any surface capable of having an agent attached thereto and includes, without limitation, metals, glass, plastics, co-polymers, colloids, lipids, cell surfaces, and the like. Essentially any surface that is capable of retaining an agent bound or attached thereto. A prototypical example of a surface used herein, is a particle such as a bead.

The term “histocompatibility” refers to the similarity of tissue between different individuals. The level of histocompatibility describes how well matched the patient and donor are. The major histocompatibility determinants are the human leukocyte antigens (HLA). HLA typing is performed between the potential donor and the potential recipient to determine how close a HLA match the two are. The closer the match the less the donated T cells and the patient's body will react against each other.

The term “human leukocyte antigens” or “HLA”, refers to proteins (antigens) found on the surface of white blood cells and other tissues that are used to match donor and patient. For instances, a patient and potential donor may have their white blood cells tested for such HLA antigens as, HLA-A, B and DR. Each individual has two sets of these antigens, one set inherited from each parent. For this reason, it is much more likely for a brother or sister to match the patient than an unrelated individual, and much more likely for persons of the same racial and ethnic backgrounds to match each other.

In a transplantation or DLI setting, the word “match” relates to how similar the HLA typing is between the donor and the recipient. The best kind of match is an “identical match”. This means that all six of the HLA antigens (2 A antigens, 2 B antigens and 2 DR antigens) are the same between the donor and the recipient. This type of match is described as a “6 of 6” match. Donors and recipients who are “mismatched” at one antigen are considered a “5 of 6” match, and so forth.

The term “allogeneic donor cells” refers to cells which are derived from an individual other than the recipient. In this regard, allogeneic donor cells can be derived from an individual who is a family member (other than an identical twin) or from an individual unrelated to the recipient.

The term “xenogenic” as used herein includes cells from a different species, including any mammal other than human, such as pig, nonhuman primate, etc. Thus, in methods described herein, the donor can be from the same species as the subject, or from a different species. In xenogeneic methods, the recipient is a mammal, such as a nonhuman primate. The donor mammal can be, by way of example, a swine, e.g., a miniature swine, or a nonhuman primate.

The term graft-versus-leukemia (GVL) or graft-versus-tumor (GVT) effect as used herein refers to an anti-leukemia or anti-tumor effect mediated by the donor cells (see e.g., A. J. Barrett, Stem Cells, Vol. 15, No. 4, 248-258, July 1997). GVL can be measured using techniques known in the art including a variety of in vitro T cell assays as well as known clinical evaluations of the recipient and molecular diagnostic techniques (e.g., PCR-based and flow cytometry-based methods for detecting residual disease) to determine the presence or absence and/or level of malignant cells.

The present invention stems from the observation that activated T cells, such as the XCELLERATE™ T cells described herein and elsewhere (see e.g., U.S. patent application Ser. Nos. 10/762,210; 10/350,305; 10/187,467; 10/133,236; 08/253,694; 08/435,816; 08/592,711; 09/183,055; 09/350,202; and 09/252,150; and U.S. Pat. Nos. 6,352,694; 5,858,358; and 5,883,223), produce less GVHD than do naïve T cells. At the same time, these cells can be used effectively to induce a GVL effect when infused into a patient. As such, the T cells of the present invention can be used in DLI with minimal risk of GVHD while still maintaining effective GVL, or more generally, graft versus tumor, effect. Without being bound by theory, DLI is usually used in the setting of allogeneic stem cell transplant, following significant conditioning to enable the donor stem cells to engraft. Unfortunately, current methods for conditioning of patients for allogeneic BMT are particularly harsh and lead to activated inflammatory stimuli, leading to an environment that encourages the development of GVHD. Thus, the present invention is generally related to methods for treating cancer with donor lymphocyte infusions in the absence of harmful conditioning.

### Donor Cells

Generally, the donor cells of the present invention are derived from an individual other than the recipient. In this regard, the donor cells can be derived from related or unrelated individuals. In certain embodiments, the donor cells are matched donor cells. In this regard, the donor cells may be matched at all 6 HLA alleles (HLA-A, B, and DR) or at fewer HLA alleles. In a further embodiment, the donor cells are mismatched. In an additional embodiment, the donor cells are derived from a sibling, an unrelated donor or a haploidentical donor. As would be recognized by the skilled artisan, HLA typing can be performed using any of a variety of techniques known in the art and can also be carried out by a certified molecular diagnostic laboratory. Donors can also be identified through marrow registries. In certain embodiments, the donor cells can be xenogeneic and can be derived from any mammal, such as nonhuman primates, pigs, etc.

The methods described herein can be used where, as between the donor and recipient, there is any degree of mismatch at MHC loci or other loci which influence graft rejection. Unlike conventional bone marrow transplantation, mismatch is desirable in methods of the invention, as mismatch promotes GVL effects. Methods of the invention can be used where, as between allogeneic donor and recipient, there is a mismatch at least one MHC locus or at least one other locus that mediates recognition and rejection, e.g., a minor antigen locus. With respect to class I and class II MHC loci, the donor and recipient can be: matched at class I and mismatched at class II; mismatched at class I and matched at class II; mismatched at class I and mismatched at class II; matched at class I, matched at class II. Mismatched, at class I or II, can mean mismatched at one or two haplotypes. Mismatched at MHC class I means mismatched for one or more MHC class I loci, e.g., in the case of humans, mismatched at one or more of HLA-A, HLA-B, or HLA-C. Mismatched at MHC class II means mismatched at one or more MHC class II loci, e.g., in the case of humans, mismatched at one or more of a DP-α, a DP-β, a DQ-α, a DQ-β, a DR-α, or a DR-β. In any of these combinations other loci which control recognition and rejection, e.g., minor antigen loci, can be matched or mismatched. In certain embodiments, it is desirable to have a mismatch at least one class I or class II locus and, in other embodiments, a mismatch at one class I and one class II locus.

The methods described herein for inducing tolerance to an allogeneic antigen or allogeneic graft can be used where, as between the donor and recipient, there is any degree of reactivity in a mixed lymphocyte assay, e.g., wherein there is no, low, intermediate, or high mixed lymphocyte reactivity between the donor and the recipient. In certain embodiments mixed lymphocyte reactivity is used to define mismatch for class II, and the invention includes methods for performing allogeneic grafts between individuals with any degree of mismatch at class II as defined by a mixed lymphocyte assay. Serological tests can be used to determine mismatch at class I or II loci and the invention includes methods for performing allogeneic grafts between individuals with any degree of mismatch at class I and or II as measured with serological methods. In a further, embodiment, the invention features methods for performing allogeneic grafts between individuals which, as determined by serological and or mixed lymphocyte reactivity assay, are mismatched at both class I and class II.

In one embodiments the donor and the subject are not related, e.g., the donor is not a sibling, the offspring of, or the parent of the recipient.

In one embodiment of the present invention, a bank of white blood cells is established wherein donors are HLA-typed, apheresis is carried out and the resulting cells are aliquoted and stored until needed for an appropriately matched recipient.

### T Cell Compositions

As noted above, the donor T cells of the present invention can be used with or without activation. Generally, the activated T cells of the present invention are generated by cell surface moiety ligation that induces activation. The activated T cells are generated by activating a population of T cells and stimulating an accessory molecule on the surface of the T cells with a ligand which binds the accessory molecule, referred to as the XCELLERATE™ process, as described for example, in U.S. patent application Ser. Nos. 10/762,210; 10/350,305; 10/187,467; 10/133,236; 08/253,694; 08/435,816; 08/592,711; 09/183,055; 09/350,202; and 09/252,150; and U.S. Pat. Nos. 6,352,694; 5,858,358; and 5,883,223.

T cells can be obtained from a number of sources, including peripheral blood mononuclear cells, bone marrow, cord blood, thymus, tissue biopsy, lymph node tissue, spleen tissue, or any other lymphoid tissue. T cells can also be obtained from T cell lines. T cells may also be obtained from a xenogeneic source, for example, from mouse, rat, non-human primate, and pig.

In one embodiment, cells from the circulating blood of an individual (i.e., the donor) are obtained by apheresis or leukapheresis. The apheresis product typically contains lymphocytes, including T cells, monocytes, granulocytes, B cells, other nucleated white blood cells, red blood cells, and platelets. In one embodiment, the cells collected by apheresis or leukapheresis may be washed to remove the plasma fraction and to place the cells in an appropriate buffer or media for subsequent processing steps. In one embodiment of the invention, the cells are washed with phosphate buffered saline (PBS). In an alternative embodiment, the wash solution lacks calcium and may lack magnesium or may lack many if not all divalent cations. As those of ordinary skill in the art would readily appreciate a washing step may be accomplished by methods known to those in the art, such as by using a semi-automated “flow-through” centrifuge (for example, the Cobe 2991 cell processor, Baxter) according to the manufacturer's instructions. After washing, the cells may be resuspended in a variety of biocompatible buffers, such as, for example, Ca⁺⁺/Mg⁺⁺ free PBS. Alternatively, the undesirable components of the apheresis sample may be removed and the cells directly resuspended in culture media.

In another embodiment, T cells are isolated from peripheral blood lymphocytes by lysing the red blood cells, isolating and reserving the monocytes as described previously, or for example, by centrifugation through a PERCOLL™ gradient. A specific subpopulation of T cells, such as CD28⁺, CD4⁺, CD8⁺, CD45RA⁺, and CD45RO⁺T cells, can be further isolated by positive or negative selection techniques. For example, CD3⁺, CD28⁺ T cells can be positively selected using CD3/CD28 conjugated magnetic beads (e.g., DYNABEADS™ M-450 CD3/CD28 T Cell Expander). In one aspect of the present invention, enrichment of a T cell population by negative selection can be accomplished with a combination of antibodies directed to surface markers unique to the negatively selected cells. A preferred method is cell sorting and/or selection via negative magnetic immunoadherence or flow cytometry that uses a cocktail of monoclonal antibodies directed to cell surface markers present on the cells negatively selected. For example, to enrich for CD4⁺ cells by negative selection, a monoclonal antibody cocktail typically includes antibodies to CD14, CD20, CD11b, CD16, HLA-DR, and CD8.

Accordingly, in one embodiment, the invention uses paramagnetic particles of a size sufficient to be engulfed by phagocytotic monocytes, that are subsequently removed through magnetic separation. In certain embodiments, the paramagnetic particles are commercially available beads, for example, those produced by Dynal AS under the trade name Dynabeads™. Exemplary Dynabeads™ in this regard are M-280, M-450, and M-500. In one aspect, other non-specific cells are removed by coating the paramagnetic particles with “irrelevant” proteins (e.g., serum proteins or antibodies). Irrelevant proteins and antibodies include those proteins and antibodies or fragments thereof that do not specifically target the T cells to be expanded. In certain embodiments, the irrelevant beads include beads coated with sheep anti-mouse antibodies, goat anti-mouse antibodies, and human serum albumin.

Another method to prepare the T cells for stimulation is to freeze the cells after the washing step, which does not require the monocyte-removal step. Wishing not to be bound by theory, the freeze and subsequent thaw step provides a more uniform product by removing granulocytes and, to some extent, monocytes in the cell population. After the washing step that removes plasma and platelets, the cells may be suspended in a freezing solution. While many freezing solutions and parameters are known in the art and will be useful in this context, one method involves using PBS containing 20% DMSO and 8% human serum albumin (HSA), or other suitable cell freezing media. This is then diluted 1:1 with media so that the final concentration of DMSO and HSA are 10% and 4%, respectively. The cells are then frozen to −80° C. at a rate of 1° per minute and stored in the vapor phase of a liquid nitrogen storage tank. Other methods of controlled freezing may be used as well as uncontrolled freezing immediately at −20° C. or in liquid nitrogen.

The activated T cells of the present invention are generated by cell surface moiety ligation that induces activation. The activated T cells are generated by activating a population of T cells and stimulating an accessory molecule on the surface of the T cells with a ligand which binds the accessory molecule, as described for example, in U.S. patent application Ser. Nos. 10/762,210; 10/350,305; 10/187,467; 10/133,236; 08/253,694; 08/435,816; 08/592,711; 09/183,055; 09/350,202; and 09/252,150; and U.S. Pat. Nos. 6,352,694; 5,858,358; and 5,883,223.

Generally, T cell activation may be accomplished by cell surface moiety ligation, such as stimulating the T cell receptor (TCR)/CD3 complex or the CD2 surface protein with an agent as described herein. Exemplary agents include, but are not limited to, antibodies. A number of anti-human CD3 monoclonal antibodies are commercially available, exemplary are, clone BC3 (XR-CD3; Fred Hutchinson Cancer Research Center, Seattle, Wash.), OKT3, prepared from hybridoma cells obtained from the American Type Culture Collection, and monoclonal antibody G19-4. Similarly, stimulatory forms of anti-CD2 antibodies are known and available. Stimulation through CD2 with anti-CD2 antibodies is typically accomplished using a combination of at least two different anti-CD2 antibodies. Stimulatory combinations of anti-CD2 antibodies that have been described include the following: the T11.3 antibody in combination with the T11.1 or T11.2 antibody (Meuer et al., Cell 36:897-906, 1984), and the 9.6 antibody (which recognizes the same epitope as T11.1) in combination with the 9-1 antibody (Yang et al., J. Immunol 137:1097-1100, 1986). Other antibodies that bind to the same epitopes as any of the above described antibodies can also be used. Additional antibodies, or combinations of antibodies, can be prepared and identified by standard techniques. Stimulation may also be achieved through contact with agents such as antigen, peptide, protein, peptide-MHC tetramers (see Altman, et al. Science 1996 Oct. 4; 274(5284):94-6), superantigens (e.g., Staphylococcus enterotoxin A (SEA), Staphylococcus enterotoxin B (SEB), Toxic Shock Syndrome Toxin 1 (TSST-1)), endotoxin, or through a variety of mitogens, including but not limited to, phytohemagglutinin (PHA), phorbol myristate acetate (PMA) and ionomycin, lipopolysaccharide (LPS), T cell mitogen, and IL-2.

To further activate a population of T cells, a co-stimulatory or accessory molecule on the surface of the T cells, such as CD28, is stimulated with an agent (e.g., an antibody or a ligand) that binds the accessory molecule. Accordingly, one of ordinary skill in the art will recognize that any agent, including an anti-CD28 antibody or fragment thereof capable of cross-linking the CD28 molecule, or a natural ligand for CD28, such as B7-1, can be used to stimulate T cells. Exemplary anti-CD28 antibodies or fragments thereof useful in the context of the present invention include monoclonal antibody 9.3 (IgG2ₐ) (Bristol-Myers Squibb, Princeton, N.J.), monoclonal antibody KOLT-2 (IgG1), 15E8 (IgG1), 248.23.2 (IgM), clone B-T3 (XR-CD28; Diaclone, Besanøon, France) and EX5.3D10 (IgG2ₐ) (ATCC HB11373). Exemplary natural ligands include the B7 family of proteins, such as B7-1 (CD80) and B7-2 (CD86) (Freedman et al., J. Immunol. 137:3260-3267, 1987; Freeman et al., J. Immunol. 143:2714-2722, 1989; Freeman et al., J. Exp. Med. 174:625-631, 1991; Freeman et al., Science 262:909-911, 1993; Azuma et al., Nature 366:76-79, 1993; Freeman et al., J. Exp. Med. 178:2185-2192, 1993).

In addition, binding homologues of a natural ligand, whether native or synthesized by chemical or recombinant techniques, can also be used in accordance with the present invention. Other agents may include natural and synthetic ligands. Agents may include, but are not limited to, other antibodies or fragments thereof, a peptide, polypeptide, growth factor, cytokine, chemokine, glycopeptide, soluble receptor, steroid, hormone, mitogen, such as PHA, or other superantigens.

The methods of the present invention preferably use agents/ligands bound to a surface. The surface may be any surface capable of having an agent bound thereto or integrated into and that is biocompatible, that is, substantially non-toxic to the target cells to be stimulated. The biocompatible surface may be biodegradable or non-biodegradable. The surface may be natural or synthetic, and a synthetic surface may be a polymer. The surface may comprise collagen, purified proteins, purified peptides, polysaccharides, glycosaminoglycans, or extracellular matrix compositions. A polysaccharide may include for example, cellulose, agarose, dextran, chitosan, hyaluronic acid, or alginate. Other polymers may include polyesters, polyethers, polyanhydrides, polyalkylcyanoacryllates, polyacrylamides, polyorthoesters, polyphosphazenes, polyvinylacetates, block copolymers, polypropylene, polytetrafluorethylene (PTFE), or polyurethanes. The polymer may be lactic acid or a copolymer. A copolymer may comprise lactic acid and glycolic acid (PLGA). Non-biodegradable surfaces may include polymers, such as poly(dimethylsiloxane) and poly(ethylene-vinyl acetate). Biocompatible surfaces include for example, glass (e.g., bioglass), collagen, metal, hydroxyapatite, aluminate, bioceramic materials, hyaluronic acid polymers, alginate, acrylic ester polymers, lactic acid polymer, glycolic acid polymer, lactic acid/glycolic acid polymer, purified proteins, purified peptides, or extracellular matrix compositions. Other polymers comprising a surface may include glass, silica, silicon, hydroxyapatite, hydrogels, collagen, acrolein, polyacrylamide, polypropylene, polystyrene, nylon, or any number of plastics or synthetic organic polymers, or the like. The surface may comprise a biological structure, such as a liposome or a cell. The surface may be in the form of a lipid, a plate, bag, pellet, fiber, mesh, or particle. A particle may include, a colloidal particle, a microsphere, nanoparticle, a bead, or the like. In the various embodiments, commercially available surfaces, such as beads or other particles, are useful (e.g., Miltenyi Particles, Miltenyi Biotec, Germany; Sepharose beads, Pharmacia Fine Chemicals, Sweden; DYNABEADS™, Dynal Inc., New York; PURABEADS™, Prometic Biosciences).

When beads are used, the bead may be of any size that effectuates target cell stimulation. In one embodiment, beads are preferably from about 5 nanometers to about 500 μm in size. Accordingly, the choice of bead size depends on the particular use the bead will serve. For example, if the bead is used for monocyte depletion, a small size is chosen to facilitate monocyte ingestion (e.g., 2.8 μm and 4.5 μm in diameter or any size that may be engulfed, such as nanometer sizes); however, when separation of beads by filtration is desired, bead sizes of no less than 50 μm are typically used. Further, when using paramagnetic beads, the beads typically range in size from about 2.8 μm to about 500 μm and more preferably from about 2.8 μm to about 50 μm. Lastly, one may choose to use super-paramagnetic nanoparticles which can be as small as about 10⁻⁵ nm. Accordingly, as is readily apparent from the discussion above, virtually any particle size may be utilized.

An agent may be attached or coupled to, or integrated into a surface by a variety of methods known and available in the art. The agent may be an antibody, a natural ligand, a protein ligand, or a synthetic ligand. The attachment may be covalent or noncovalent, electrostatic, or hydrophobic and may be accomplished by a variety of attachment means, including for example, chemical, mechanical, enzymatic, electrostatic, or other means whereby an agent is capable of stimulating the cells. For example, the antibody to a ligand first may be attached to a surface, or avidin or streptavidin may be attached to the surface for binding to a biotinylated ligand. The antibody to the ligand may be attached to the surface via an anti-idiotype antibody. Another example includes using protein A or protein G, or other non-specific antibody binding molecules, attached to surfaces to bind an antibody. Alternatively, the ligand may be attached to the surface by chemical means, such as cross-linking to the surface, using commercially available cross-linking reagents (Pierce, Rockford, Ill.) or other means. In certain embodiments, the ligands are covalently bound to the surface. Further, in one embodiment, commercially available tosyl-activated DYNABEADS™ or DYNABEADS™ with epoxy-surface reactive groups are incubated with the polypeptide ligand of interest according to the manufacturer's instructions. Briefly, such conditions typically involve incubation in a phosphate buffer from pH 4 to pH 9.5 at temperatures ranging from 4 to 37 degrees C.

In one aspect, the agent, such as certain ligands may be of singular origin or multiple origins and may be antibodies or fragments thereof while in another aspect, when utilizing T cells, the co-stimulatory ligand is a B7 molecule (e.g., B7-1, B7-2). These ligands are coupled to the surface by any of the different attachment means discussed above. The B7 molecule to be coupled to the surface may be isolated from a cell expressing the co-stimulatory molecule, or obtained using standard recombinant DNA technology and expression systems that allow for production and isolation of the co-stimulatory molecule(s) as described herein. Fragments, mutants, or variants of a B7 molecule that retain the capability to trigger a co-stimulatory signal in T cells when coupled to the surface of a cell can also be used. Furthermore, one of ordinary skill in the art will recognize that any ligand useful in the activation and induction of proliferation of a subset of T cells may also be immobilized on beads or culture vessel surfaces or any surface. In addition, while covalent binding of the ligand to the surface is one preferred methodology, adsorption or capture by a secondary monoclonal antibody may also be used. The amount of a particular ligand attached to a surface may be readily determined by flow cytometric analysis if the surface is that of beads or determined by enzyme-linked immunosorbent assay (ELISA) if the surface is a tissue culture dish, mesh, fibers, bags, for example.

In a particular embodiment, the stimulatory form of a B7 molecule or an anti-CD28 antibody or fragment thereof is attached to the same solid phase surface as the agent that stimulates the TCR/CD3 complex, such as an anti-CD3 antibody. In an additional embodiment, the stimulatory form of a 4-1BB molecule or an anti-4-1BB antibody or fragment thereof is attached to the same solid phase surface as the agent that stimulates the TCR/CD3 complex, such as an anti-CD3 antibody. In addition to anti-CD3 antibodies, other antibodies that bind to receptors that mimic antigen signals may be used. For example, the beads or other surfaces may be coated with combinations of anti-CD2 antibodies and a B7 molecule and in particular anti-CD3 antibodies and anti-CD28 antibodies. In further embodiments, the surfaces may be coated with three or more agents, such as combinations of any of the agents described herein, for example, anti-CD3 antibodies, anti-CD28 antibodies, and anti-4-1BB antibodies.

The primary stimulatory signal and the co-stimulatory signal for the T-cell may be provided by different protocols. For example, the agents providing each signal may be in solution or coupled to a surface. When coupled to a surface, the agents may be coupled to the same surface (i.e., in “cis” formation) or to separate surfaces (i.e., in “trans” formation). Alternatively, one agent may be coupled to a surface and the other agent in solution. In another embodiment, surfaces may be coated or conjugated directly (including covalently) or indirectly (e.g., streptavidin/biotin and the like) with antibodies or other components to stimulate T cell activation and expansion. In one embodiment, the agent providing the co-stimulatory signal is bound to a cell surface and the agent providing the primary activation signal is in solution or coupled to a surface. In certain embodiments, both agents can be in solution. In another embodiment, the agents may be in soluble form, and then cross-linked to a surface, such as a cell expressing FC receptors or an antibody or other binding agent which will bind to the agents. In one embodiment, the two agents are immobilized or otherwise attached on beads, either on the same bead, i.e., “cis,” or to separate beads, i.e., “trans.” By way of example, the agent providing the primary activation signal is an anti-CD3 antibody and the agent providing the co-stimulatory signal is an anti-CD28 antibody; and both agents are co-immobilized or otherwise attached to the same bead in equivalent molecular amounts. In one embodiment, a 1:1 ratio of each antibody bound to the beads for CD4⁺ T-cell expansion and T-cell growth is used. In certain aspects of the present invention, a ratio of anti CD3:CD28 antibodies bound to the beads is used such that an increase in T cell expansion is observed as compared to the expansion observed using a ratio of 1:1. In one particular embodiment an increase of from about 0.5 to about 3 fold is observed as compared to the expansion observed using a ratio of 1:1. In one embodiment, the ratio of CD3:CD28 antibody bound to the beads ranges from 100:1 to 1:100 and all integer values there between. In one aspect of the present invention, more anti-CD28 antibody is bound to the particles than anti-CD3 antibody, i.e. the ratio of CD3:CD28 is less than one. In certain embodiments of the invention, the ratio of anti CD28 antibody to anti CD3 antibody bound to the beads is greater than 2:1. In one particular embodiment, a 1:200 CD3:CD28 ratio of antibody bound to beads is used. In one particular embodiment, a 1:100 CD3:CD28 ratio of antibody bound to beads is used. In another embodiment, a 1:75 CD3:CD28 ratio of antibody bound to beads is used. In a further embodiment, a 1:50 CD3:CD28 ratio of antibody bound to beads is used. In another embodiment, a 1:30 CD3:CD28 ratio of antibody bound to beads is used. In one preferred embodiment, a 1:10 CD3:CD28 ratio of antibody bound to beads is used. In another embodiment, a 1:3 CD3:CD28 ratio of antibody bound to the beads is used. In yet another embodiment, a 3:1 CD3:CD28 ratio of antibody bound to the beads is used.

In certain aspects of the present invention, three or more agents are coupled to a surface. In certain embodiments, the agents may be coupled to the same surface (i.e., in “cis” formation) or to separate surfaces (i.e., in “trans” formation). Alternatively, one or more agents may be coupled to a surface and the other agent or agents may be in solution.

Ratios of particles to cells from 1:500 to 500:1 and any integer values in between may be used to stimulate T-cells or other target cells. As those of ordinary skill in the art can readily appreciate, the ratio of particle to cells may dependant on particle size relative to the target cell. For example, small sized beads could only bind a few cells, while larger beads could bind many. In certain embodiments the ratio of cells to particles ranges from 1:100 to 100:1 and any integer values in-between and in further embodiments the ratio comprises 1:9 to 9:1 and any integer values in between, can also be used to stimulate T-cells. The ratio of anti-CD3- and anti-CD28-coupled beads particles to T-cells that result in T-cell stimulation can vary as noted above, however in certain embodiments, the ratio of anti-CD3 and anti-CD28 coupled beads to cells includes 1:50, 1:40, 1:30, 1:20, 1:15, 1:10, 1:5, 1:4, 1:3, 1:2, 1:1, 2:1, 3:1, 4:1 to 6:1, with one particular ratio being 3:1 beads/particles per T-cell. In one embodiment, a ratio of particles to cells of 1:1 or less is used. In further embodiments, the ratio of particles to cells can be varied depending on the day of stimulation. For example, in one embodiment, the ratio of particles to cells is from 1:1 to 10:1 on the first day and additional particles are added to the cells every day or every other day thereafter for up to 10 days, at final ratios of from 1:1 to 1:10 (based on cell counts on the day of addition). In one particular embodiment, the ratio of particles to cells is 1:1 on the first day of stimulation and adjusted to 1:5 on the third and fifth days of stimulation. In another embodiment, particles are added on a daily or every other day basis to a final ratio of 1:1 on the first day, and 1:5 on the third and fifth days of stimulation. In another embodiment, the ratio of particles to cells is 2:1 on the first day of stimulation and adjusted to 1:10 on the third and fifth days of stimulation. In another embodiment, particles are added on a daily or every other day basis to a final ratio of 1:1 on the first day, and 1:10 on the third and fifth days of stimulation. One of skill in the art will appreciate that a variety of other ratios may be suitable for use in the present invention. In particular, ratios will vary depending on particle size and on cell size and type.

Using certain methodologies it may be advantageous to maintain long-term stimulation of a population of T-cells following the initial activation and stimulation, by separating the T-cells from the stimulus after a period of about 12 to about 14 days. The rate of T-cell proliferation is monitored periodically (e.g., daily) by, for example, examining the size or measuring the volume of the T-cells, such as with a Coulter Counter. In this regard, a resting T-cell has a mean diameter of about 6.8 microns, and upon initial activation and stimulation, in the presence of the stimulating ligand, the T-cell mean diameter will increase to over 12 microns by day 4 and begin to decrease by about day 6. When the mean T-cell diameter decreases to approximately 8 microns, the T-cells may be reactivated and re-stimulated to induce further proliferation of the T-cells. Alternatively, the rate of T-cell proliferation and time for T-cell re-stimulation can be monitored by assaying for the presence of cell surface molecules, such as, CD154, CD54, CD25, CD137, CD134, B7-1, B7-2, which are induced on activated T-cells.

For inducing long-term stimulation of a population of CD4⁺ and/or CD8⁺ T-cells, it may be necessary to reactivate and re-stimulate the T-cells with a stimulatory agent such as an anti-CD3 antibody and an anti-CD28 antibody (such as B-T3, XR-CD28 (Diaclone, Besanøon, France) or monoclonal antibody ES5.2D8 several times to produce a population of CD4⁺ or CD8⁺ cells increased in number from about 10 to about 1,000-fold the original T-cell population. Thus, for example, in one embodiment of the present invention, T-cells are stimulated as described herein for 2-3 rounds of stimulation. In further embodiments, T-cells are stimulated as described herein for 4 or 5 rounds of stimulation.

In another embodiment, the time of exposure to stimulatory agents such as anti-CD3/anti-CD28 (i.e., CD3xCD28)-coated beads may be modified or tailored to obtain a desired T-cell phenotype. One may desire a greater population of helper T-cells (T_{H}), typically CD4⁺ as opposed to CD8⁺ cytotoxic or suppressor T-cells (T_{C}), because an expansion of TH cells could induce desired GVL. CD4⁺ T-cells, express important immune-regulatory molecules, such as GM-CSF, CD40L, and IL-2, for example. Where CD4-mediated help is preferred, a method, such as that described herein, which preserves or enhances the CD4:CD8 ratio could be of significant benefit. In one aspect of the present invention, it may be beneficial to increase the number of infused cells expressing GM-CSF, or IL-2, all of which are expressed predominantly by CD4⁺ T-cells. Alternatively, in situations where CD4-help is needed less and increased numbers of CD8⁺ T-cells are desirous, the T cell activation approaches described herein can also be utilized, by for example, pre-selecting for CD8⁺ cells prior to stimulation and/or culture. Such situations may exist where increased levels of IFN-γ is preferred. Further, in other applications, it may be desirable to utilize a population of T_{H}1-type cells versus T_{H}2-type cells (or vice versa).

To effectuate isolation of different T-cell populations, times of cell surface moiety ligation that induces activation may be varied or pulsed. For example expansion times may be varied to obtain the specific phenotype of interest and/or different types of stimulatory agents may be used (e.g., antibodies or fragments thereof, a peptide, polypeptide, MHC/peptide tetramer, growth factor, cytokine, chemokine, glycopeptide, soluble receptor, steroid, hormone, mitogen, such as PHA, or other superantigens). The expression of a variety of phenotypic markers change over time; therefore, a particular time point or stimulatory agent may be chosen to obtain a specific population of T-cells. Accordingly, depending on the cell type to be stimulated, the stimulation and/or expansion time may be four weeks or less, 2 weeks or less, 10 days or less, or 8 days or less (four weeks or less includes all time ranges from 4 weeks down to 1 day (24 hours)). In some embodiments, stimulation and expansion may be carried out for 6 days or less, 4 days or less, 2 days or less, and in other embodiments for as little as 24 or less hours, and preferably 4-6 hours or less (these ranges include any integer values in between). When stimulation of T-cells is carried out for shorter periods of time, the population of T-cells may not increase in number as dramatically, but the population will provide more robust and healthy activated T-cells that can continue to proliferate in vivo and more closely resemble the natural effector T-cell pool.

In another embodiment, the time of exposure to stimulatory agents such as anti-CD3/anti-CD28 (i.e., 3×28)-coated beads may be modified or tailored to obtain a desired T cell phenotype. Alternatively, a desired population of T cells can be selected using any number of selection techniques, prior to stimulation. One may desire a greater population of helper T cells (TH), typically CD4⁺ as opposed to CD8⁺ cytotoxic or regulatory T cells, because an expansion of TH cells could improve or restore overall immune responsiveness. While many specific immune responses are mediated by CD8⁺ antigen-specific T cells, which can directly lyse or kill target cells, most immune responses require the help of CD4⁺ T cells, which express important immune-regulatory molecules, such as GM-CSF, CD40L, and IL-2, for example. Where CD4-mediated help if preferred, a method, such as that described herein, which preserves or enhances the CD4:CD8 ratio could be of significant benefit. Increased numbers of CD4⁺ T cells can increase the amount of cell-expressed CD40L introduced into patients, potentially improving target cell visibility (improved APC function). Similar effects can be seen by increasing the number of infused cells expressing GM-CSF, or IL-2, all of which are expressed predominantly by CD4⁺ T cells. Alternatively, in situations where CD4-help is needed less and increased numbers of CD8⁺ T cells are desirous, the XCELLERATE approaches described herein can also be utilized, by for example, pre-selecting for CD8⁺ cells prior to stimulation and/or culture. Such situations may exist where increased levels of IFN-γ or increased cytolysis of a target cell is preferred. In a further embodiment, the XCELLERATE™ process can be modified or tailored to promote homing of T cells to particular sites of interest, such as lymph nodes or sites of inflammation, or to bone marrow, for example. Additionally, in certain embodiments, the XCELLERATE approaches described herein can also be utilized for the generation of T regulatory cells for specific immunosuppression in the case of inflammatory disease, autoimmunity, and foreign graft acceptance, or any other disease setting where regulatory T cells are desired. Classically, T regulatory cells have a CD4⁺, CD25⁺, CD62L^{hi}, GITR⁺, and FoxP3⁺ phenotype (see for example, Woo, et al., J Immunol. 2002 May 1; 168(9):4272-6; Shevach, E. M., Annu. Rev. Immunol. 2000, 18:423; Stephens, et al., Eur. J. Immunol. 2001, 31:1247; Salomon, et al, Immunity 2000, 12:431; and Sakaguchi, et al., Immunol. Rev. 2001, 182:18). Regulatory T cells can be generated and expanded using the methods of the present invention. The regulatory T cells can be antigen-specific and/or polyclonal. Regulatory T cells can also be generated using art-recognized techniques as described for example, in Woo, et al.; Shevach, E. M.; Stephens, et al.; Salomon, et al.; and Sakaguchi, et al.; Supra.

Further, in addition to CD4 and CD8 markers, other phenotypic markers vary significantly, but in large part, reproducibly during the course of the cell expansion process. Thus, such reproducibility enables the ability to tailor an activated T-cell product for specific purposes.

In one such example, among the important phenotypic markers that reproducibly vary with time are the high affinity IL-2 receptor (CD25), CD40 ligand (CD154), and CD45RO (a molecule that by preferential association with the TCR may increase the sensitivity of the TCR to antigen binding). As one of ordinary skill in the art readily appreciates, such molecules are important for a variety of reasons. For example, CD25 constitutes an important part of the autocrine loop that allows rapid T-cell division. CD154 has been shown to play a key role in stimulating maturation of the antigen-presenting dendritic cells; activating B-cells for antibody production; regulating T_{H} cell proliferation; enhancing T_{C} cell differentiation; regulating cytokine secretion of both T_{H} cells and antigen-presenting cells; and stimulating expression of co-stimulatory ligands, including CD80, CD86, and CD154.

In addition to the cytokines and the markers discussed previously, expression of adhesion molecules known to be important for mediation of T-cell activation and immune-mediated modulation of target cells also change dramatically but reproducibly over the course of the ex vivo expansion process. For example, CD62L is important for homing of T-cells to lymphoid tissues and trafficking T-cells to sites of inflammation. Because down-regulation of CD62L occurs early following activation, the T-cells could be expanded for shorter periods of time. Conversely, longer periods of time in culture would generate a T-cell population with higher levels of CD62L and thus a higher ability to target the activated T-cells to these sites under other preferred conditions. Another example of a polypeptide whose expression varies over time is CD49d, an adhesion molecule that is involved in trafficking lymphocytes from blood to tissues spaces at sites of inflammation. Binding of the CD49d ligand to CD49d also allows the T-cell to receive co-stimulatory signals for activation and proliferation through binding by VCAM-1 or fibronectin ligands. The expression of the adhesion molecule CD54, involved in T-cell-APC and T-cell-T-cell interactions as well as homing to sites of inflammation, also changes over the course of expansion. Accordingly, T-cells could be stimulated for selected periods of time that coincide with the marker profile of interest and subsequently collected and infused. Activated T cells could also be applied directly to an injury site. Thus, T-cell populations could be tailored to express the markers believed to provide the most therapeutic benefit for the indication to be treated.

In the various embodiments, one of ordinary skill in the art understands removal of the stimulation signal from the cells is dependent upon the type of surface used. For example, if paramagnetic beads are used, then magnetic separation is the feasible option. Separation techniques are described in detail by paramagnetic bead manufacturers' instructions (for example, DYNAL Inc., Oslo, Norway). Furthermore, filtration may be used if the surface is a bead large enough to be separated from the cells. In addition, a variety of transfusion filters are commercially available, including 20 micron and 80 micron transfusion filters (Baxter). Accordingly, so long as the beads are larger than the mesh size of the filter, such filtration is highly efficient. In a related embodiment, the beads may pass through the filter, but cells may remain, thus allowing separation.

Although the antibodies used in the methods described herein can be readily obtained from public sources, such as the ATCC, antibodies to T-cell accessory molecules and the CD3 complex can be produced by standard techniques. Methodologies for generating antibodies for use in the methods of the invention are well-known in the art.

In one aspect of the present invention, the T cells may be genetically modified using any number of methods known in the art. For example, the T cells may be genetically modified to introduce a “suicide gene or other molecule that allows for subsequence drug/chemical removal (“killing”) of the infused donor T cells if desired. The T cells may be transfected using numerous RNA or DNA expression vectors known to those of ordinary skill in the art. Genetic modification may comprise RNA or DNA transfection using any number of techniques known in the art, for example electroporation (using e.g., the Gene Pulser II, BioRad, Richmond, Calif.), various cationic lipids, (LIPOFECTAMINE™, Life Technologies, Carlsbad, Calif.), or other techniques such as calcium phosphate transfection as described in Current Protocols in Molecular Biology, John Wiley & Sons, New York. N.Y. For example, 5-50 μg of RNA or DNA in 500 μl of Opti-MEM can be mixed with a cationic lipid at a concentration of 10 to 100 μg, and incubated at room temperature for 20 to 30 minutes. Other suitable lipids include LIPOFECTIN™, LIPOFECTAMINE™. The resulting nucleic acid-lipid complex is then added to 1-3×10⁶ cells, preferably 2×10⁶, antigen-presenting cells in a total volume of approximately 2 ml (e.g., in Opti-MEM), and incubated at 37° C. for 2 to 4 hours. The T cells may also be transduced using viral transduction methodologies as described below

The T cells may alternatively be genetically modified using retroviral transduction technologies. In one aspect of the invention, the retroviral vector may be an amphotropic retroviral vector, preferably a vector characterized in that it has a long terminal repeat sequence (LTR), e.g., a retroviral vector derived from the Moloney murine leukemia virus (MoMLV), myeloproliferative sarcoma virus (MPSV), murine embryonic stem cell virus (MESV), murine stem cell virus (MSCV), spleen focus forming virus (SFFV), or adeno-associated virus (AAV). Most retroviral vectors are derived from murine retroviruses. Retroviruses adaptable for use in accordance with the present invention can, however, be derived from any avian or mammalian cell source. These retroviruses are preferably amphotropic, meaning that they are capable of infecting host cells of several species, including humans. In one embodiment, the gene to be expressed replaces the retroviral gag, pol and/or env sequences. A number of illustrative retroviral systems have been described (e.g., U.S. Pat. Nos. 5,219,740; 6,207,453; 5,219,740; Miller and Rosman (1989) BioTechniques 7:980-990; Miller, A. D. (1990) Human Gene Therapy 1:5-14; Scarpa et al. (1991) Virology 180:849-852; Burns et al. (1993) Proc. Natl. Acad. Sci. USA 90:8033-8037; and Boris-Lawrie and Temin (1993) Cur. Opin. Genet. Develop. 3:102-109.

### Methods of Use

The present invention provides T cell compositions for use in the treatment of cancer. In particular, the present invention provides compositions of allogeneic donor cells for use in donor lymphocyte infusions (DLI) for the treatment of cancer. Generally, the goal of DLI in this context is not to achieve final engraftment; the desired effect is the GVL prior to rejection of the donor cells. In this regard, the present invention stems in part from the observation that activated T cells, such as the XCELLERATE™ T cells described herein and elsewhere, generate less GVHD than do naïve T cells while maintaining effective GVL reactivity. The percentage of alloreactive cells is lower in XCELLERATE™ T cells. Further, harsh conditioning regimens contribute to GVHD by producing inflammatory stimuli. Thus, the present invention provides for DLI for the treatment of cancer in the absence of or with greatly reduced conditioning, with or without stem cell transplant.

CD154 is expressed on activated T cells in a temporal manner and has been shown to be a key element in T cells interactions via CD40 on APCs (see e.g., U.S. application Ser. No. 10/762,210). Blocking the interaction of these two receptors can effectively alter, and even shut-off, an immune response. The activated T cells of the present invention have been shown to express elevated levels of CD40L that peak at about day 3 to day 4 of activation/expansion and remains elevated out to day 6 and day 7. Thus, without being bound by theory, high expression of CD40L may be critical in the allogeneic DLI setting and thus the T cells described herein may be particularly suited to generating an effective GVL reaction.

In certain embodiments of the present invention, the DLI are administered in the absence of any bone marrow transplant. In certain embodiments the T cells used in DLI are not activated T cells but simply donor T cells that have not been activated in vitro. In certain embodiments, the present invention also provides autologous T cell compositions.

Donor allogeneic or xenogeneic T cells can be stimulated and expanded as described herein or using other methods known in the art wherein T cells are stimulated and expanded to therapeutic levels, for the treatment of a variety of cancers. T cells of the present invention are useful for treating melanoma, non-Hodgkin's lymphoma, cutaneous T cell lymphoma, Hodgkin's disease, leukemia, plasmocytoma, sarcoma, glioma, thymoma, breast cancer, prostate cancer, colo-rectal cancer, kidney cancer, renal cell carcinoma, uterine cancer, pancreatic cancer, esophageal cancer, brain cancer, lung cancer, ovarian cancer, cervical cancer, testicular cancer, gastric cancer, esophageal cancer, multiple myeloma, hepatoma, acute lymphoblastic leukemia (ALL), acute myelogenous leukemia (AML), chronic myelogenous leukemia (CML), and chronic lymphocytic leukemia (CLL), or other cancers.

In a further embodiment, the donor T cell compositions of the present invention can be used in conjunction with a variety of anti-tumor treatment modalities, including but not limited to, GLIVEC. In this regard, one setting where the present invention can be used is in the setting where patients have been treated to minimal residual disease, for example, after chemotherapy in CLL or NHL or after GLIVEC treatment in CML. For example, patients who have less than three logs reduction of tumor cells after GLIVEC treatment or more than 0.1% tumor cells, will relapse. Thus, these patients will benefit from treatment with the T cells of the present invention. Thus, certain goals of treatment with the cells of the present invention are to clear minimal residual disease, achieve durable remission and also to allow patients to stop taking drugs without which patients relapse.

Thus, in certain embodiments of the present invention, cells activated and expanded using the methods described herein, or other methods known in the art where T cells are expanded to therapeutic levels, are administered to a patient in conjunction with (e.g. before, simultaneously or following) any number of relevant treatment modalities, including but not limited to treatment with agents such as antiviral agents, chemotherapy, radiation, immunosuppressive agents, such as cyclosporin, azathioprine, methotrexate, mycophenolate, and FK506, antibodies, or other immunoablative agents such as CAMPATH, anti-CD3 antibodies or other antibody therapies, cytoxin, fludaribine, cyclosporin, FK506, rapamycin, mycophenolic acid, steroids, FR901228, cytokines, and irradiation. These drugs inhibit either the calcium dependent phosphatase calcineurin (cyclosporine and FK506) or inhibit the p70S6 kinase that is important for growth factor induced signaling (rapamycin). (Liu et al., Cell 66:807-815, 1991; Henderson et al., Immun. 73:316-321, 1991; Bierer et al., Curr. Opin. Immun. 5:763-773, 1993; Isoniemi (supra)). In a further embodiment, the cell compositions of the present invention are administered to a patient in conjunction with (e.g. before, simulataneously or following) bone marrow transplantation, T cell ablative therapy using either chemotherapy agents such as, fludarabine, external-beam radiation therapy (XRT), cyclophosphamide, or antibodies such as OKT3 or CAMPATH. In another embodiment, the cell compositions of the present invention are administered following B-cell ablative therapy such as agents that react with CD20, e.g. Rituxan. In an additional embodiment, expanded cells are administered before or following surgery.

The dosage of the above treatments to be administered to a patient will vary with the precise nature of the condition being treated and the recipient of the treatment. The scaling of dosages for human administration can be performed according to art-accepted practices.

### Formulations/Pharmaceutical Compositions

The present invention further provides pharmaceutical compositions comprising the activated T cells and a pharmaceutically acceptable carrier. Compositions of the present invention may be administered either alone, or as a pharmaceutical composition in combination with diluents and/or with other components such as IL-2 or other cytokines or cell populations. Briefly, pharmaceutical compositions of the present invention may comprise a target cell population as described herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as ethylenediaminetetraacetic acid (EDTA) or glutathione; adjuvants (e.g., aluminum hydroxide); and preservatives. Compositions of the present invention are, in certain aspects, formulated for intravenous administration.

Pharmaceutical compositions of the present invention may be administered in a manner appropriate to the disease to be treated (or prevented). The quantity and frequency of administration will be determined by such factors as the condition of the patient, and the type and severity of the patient's disease, although appropriate dosages may be determined by clinical trials. As such, the compositions of the present invention can be administered in multiple, sequential dosages as determined by a clinician. In this regard, in certain embodiments, T cells from different donors can be used in successive cycles of treatments to reduce the risk of rejection of the infused cells. Further, in another embodiment, allogeneic cells as described herein can be administered before, at the same time, or after autologous T cell therapy. In this regard, autologous cells are activated in the same manner as allogeneic cells as described herein.

When “a therapeutically effective amount” is indicated, the precise amount of the compositions of the present invention to be administered can be determined by a physician with consideration of individual differences in age, weight, tumor size, extent of infection or metastasis, and condition of the patient. Typically, in adoptive immunotherapy studies, activated T cells are administered approximately at 2×10⁹ to 2×10¹¹ cells to the patient. (See, e.g., U.S. Pat. No. 5,057,423). In some aspects of the present invention, particularly in the use of allogeneic or xenogeneic cells, lower numbers of cells, in the range of 10⁶/kilogram (10⁶-10¹¹ per patient) may be administered. T cell, or other altered post co-culture cell compositions may be administered multiple times at dosages within these ranges. The activated T cells may be autologous or heterologous to the patient undergoing therapy.

In certain embodiments, the T cell compositions can be administered in conjunction with (e.g., before, at the same time as, or after) any of a variety of factors to encourage T cell growth in vivo, such as IL-2 and/or IL-15 or other cytokines.

In certain embodiments, the donor lymphocytes of the present invention can be administered in conjunction with stem cells (e.g., before, at the same time as or after stem cell therapy).

The administration of the subject pharmaceutical compositions may be carried out in any convenient manner, including by aerosol inhalation, injection, ingestion, transfusion, implantation or transplantation. The compositions of the present invention may be administered to a patient subcutaneously, intradermally, intramuscularly, by intravenous (i.v.) injection, or intraperitoneally. The T cell compositions of the present invention are preferably administered by i.v. injection. The compositions of activated T cells may be injected directly into a site of tissue injury.

In yet another embodiment, the pharmaceutical composition can be delivered in a controlled release system. In one embodiment, a pump may be used (see Langer, 1990, Science 249:1527-1533; Sefton 1987, CRC Crit. Ref. Biomed. Eng. 14:201; Buchwald et al., 1980; Surgery 88:507; Saudek et al., 1989, N. Engl. J. Med. 321:574). In another embodiment, polymeric materials can be used (see Medical Applications of Controlled Release, 1974, Langer and Wise (eds.), CRC Pres., Boca Raton, Fla.; Controlled Drug Bioavailability, Drug Product Design and Performance, 1984, Smolen and Ball (eds.), Wiley, New York; Ranger and Peppas, 1983; J. Macromol. Sci. Rev. Macromol. Chem. 23:61; see also Levy et al., 1985, Science 228:190; During et al., 1989, Ann. Neurol. 25:351; Howard et al., 1989, J. Neurosurg. 71:105). In yet another embodiment, a controlled release system can be placed in proximity of the therapeutic target, thus requiring only a fraction of the systemic dose (see, e.g., Medical Applications of Controlled Release, 1984, Langer and Wise (eds.), CRC Pres., Boca Raton, Fla., vol. 2, pp. 115-138).

The compositions of the present invention may also be administered using any number of matrices. Matrices have been utilized for a number of years within the context of tissue engineering (see, e.g., Principles of Tissue Engineering (Lanza, Langer, and Chick (eds.)), 1997. The present invention utilizes such matrices within the novel context of acting as an artificial lymphoid organ to support, maintain, or modulate the immune system, typically through modulation of T cells. Accordingly, the present invention can utilize those matrix compositions and formulations which have demonstrated utility in tissue engineering. Accordingly, the type of matrix that may be used in the compositions, devices and methods of the invention is virtually limitless and may include both biological and synthetic matrices. In one particular example, the compositions and devices set forth by U.S. Pat. No. 5,980,889; 5,913,998; 5,902,745; 5,843,069; 5,787,900; or 5,626,561 are utilized. Matrices comprise features commonly associated with being biocompatible when administered to a mammalian host. Matrices may be formed from both natural and synthetic materials. The matrices may be non-biodegradable in instances where it is desirable to leave permanent structures or removable structures in the body of an animal, such as an implant; or biodegradable. The matrices may take the form of sponges, implants, tubes, telfa pads, fibers, hollow fibers, lyophilized components, gels, powders, porous compositions, or nanoparticles. In addition, matrices can be designed to allow for sustained release seeded cells or produced cytokine or other active agent. In certain embodiments, the matrix of the present invention is flexible and elastic, and may be described as a semisolid scaffold that is permeable to substances such as inorganic salts, aqueous fluids and dissolved gaseous agents including oxygen.

A matrix is used herein as an example of a biocompatible substance. However, the current invention is not limited to matrices and thus, wherever the term matrix or matrices appears these terms should be read to include devices and other substances which allow for cellular retention or cellular traversal, are biocompatible, and are capable of allowing traversal of macromolecules either directly through the substance such that the substance itself is a semi-permeable membrane or used in conjunction with a particular semi-permeable substance.

Compositions comprising the activated T cells as described herein can be provided as pharmaceutically acceptable formulations using formulation methods known to those of ordinary skill in the art. These formulations can be administered by standard routes. In general, the combinations may be administered by the topical, transdermal, oral, rectal or parenteral (e.g., intravenous, subcutaneous or intramuscular) route. In addition, the combinations may be incorporated into biodegradable polymers allowing for sustained release of the composition, the polymers being implanted in the vicinity of where delivery is desired, for example, at the site of tissue injury. The biodegradable polymers and their use are described, for example, in detail in Brem et al. J. Neurosurg. 74:441-446 (1991).

The dosage of the compositions will depend on the condition being treated, and other clinical factors such as weight and condition of the human or animal, the nature of the composition, and the route of administration of the composition. It is to be understood that the present invention has application for both human and veterinary use.

The formulations include those suitable for oral, rectal, ophthalmic, (including intravitreal or intracameral) nasal, topical (including buccal and sublingual), vaginal or parenteral (including subcutaneous, intramuscular, intravenous, intradermal, intratracheal, and epidural) administration. The formulations may conveniently be presented in a dosage form and may be prepared by conventional pharmaceutical techniques. Such techniques include the step of bringing into association the active ingredient and the pharmaceutical carrier(s) or excipient(s). In general, the formulations are prepared by uniformly and intimately bringing into associate the active ingredient with liquid carriers or finely divided solid carriers or both, and then, if necessary, shaping the product.

Formulations suitable for topical administration to the skin may be presented as ointments, creams, gels and pastes comprising the ingredient to be administered in a pharmaceutical acceptable carrier. A preferred topical delivery system is a transdermal patch containing the ingredient to be administered.

Formulations for rectal administration may be presented as a suppository with a suitable base comprising, for example, cocoa butter or a salicylate.

Formulations suitable for nasal administration, wherein the carrier is a solid, include a coarse powder having a particle size, for example, in the range of 20 to 500 microns which is administered in the manner in which snuff is administered, i.e., by rapid inhalation through the nasal passage from a container of the powder held close up to the nose. Suitable formulations, wherein the carrier is a liquid, for administration, as for example, a nasal spray or as nasal drops, include aqueous or oily solutions of the active ingredient.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampules and vials, and may be stored in a freeze-dried (lyophilized) conditions requiring only the addition of the sterile liquid carrier, for example, water for injections, immediately prior to use. Extemporaneous injection solutions and suspensions may be prepared from sterile powders, granules and tablets of the kind previously described.

Preferred unit dosage formulations are those containing a daily dose or unit, daily sub-dose, as herein above recited, or an appropriate fraction thereof, of the administered ingredient.

It should be understood that in addition to the ingredients, particularly mentioned above, the formulations of the present invention may include other agents conventional in the art having regard to the type of formulation in question, for example, those suitable for oral administration may include flavoring agents.

In one embodiment of the present invention, compositions comprising cells of the present invention, e.g., activated T cells are targeted to the desired location through the use of paramagnetic beads and application of a magnetic force inside or outside a target tissue (as described, for example, in U.S. Pat. No. 6,203,487). Briefly, the cells of the present invention, either activated T cells or cells previously co-cultured with activated T cells, are exposed to paramagnetic beads conjugated to appropriate surface markers either in vivo or in vitro or a combination of the two such that binding of the paramagnetic particle to the cells occurs. If carried out in vitro, a composition comprising cells bound to the paramagnetic particles and a pharmaceutically acceptable excipient is administered to a mammal. A magnet may be placed adjacent to a target tissue, i.e., an area of the body or a selected tissue or organ into which local cell delivery is desired. The magnet can be positioned superficial to the body surface or can be placed internal to the body surface using surgical or percutaneous methods inside or outside the target tissue for local delivery. The magnetic particles bound to cells are delivered either by direct injection into the selected tissue or to a remote site and allowed to passively circulate to the target site or are actively directed to the target site with a magnet or the targeting ligand.

All references referred to within the text are hereby incorporated by reference in their entirety. Moreover, all numerical ranges utilized herein explicitly include all integer values within the range and selection of specific numerical values within the range is contemplated depending on the particular use.

From the foregoing it will be appreciated that, although specific embodiments of the invention have been described herein for purposes of illustration, various modifications may be made without deviating from the spirit and scope of the invention. Accordingly, the invention is not limited except as by the appended claims.

What is claimed is:

1. A method for treating cancer comprising administering allogeneic T cells to a cancer patient, wherein said patient has not received a conditioning regimen.

2. The method of claim 1 wherein said T cells are activated T cells.

3. The method of claim 2, wherein the T cells are activated by a method comprising,
contacting a population of allogeneic cells from a suitable donor, wherein at least a portion of the population comprises T cells, with a surface, wherein said surface has attached thereto a first agent which stimulates a TCR/CD3 complex-associated signal in the T cells and a second agent that binds the CD28 accessory molecule on the surface of the T cells, thereby activating the T cells.

4. The method of claim 3 wherein the first agent is an antibody or an antigen-binding fragment thereof.

5. The method of claim 4 wherein the antibody or antigen-binding fragment thereof is a monoclonal antibody or antigen-binding fragment thereof.

6. The method of claim 4 wherein the antibody is an anti-CD3 antibody.

7. The method of claim 3 wherein the second agent is an antibody or an antigen-binding fragment thereof.

8. The method of claim 7 wherein the antibody or antigen-binding fragment thereof is a monoclonal antibody or antigen-binding fragment thereof.

9. The method of claim 8 wherein the antibody is an anti-CD28 antibody.

10. The method of claim 3 wherein the first and the second agents are both antibodies or antigen-binding fragments thereof.

11. The method of claim 10 wherein the first agent is an anti-CD3 antibody or antigen-binding fragments thereof and the second agent is an anti-CD28 antibody or antigen-binding fragments thereof.

12. The method of claim 3 wherein the second agent is a natural ligand of CD28.

13. The method of claim 12 wherein the natural ligand is B7-1.

14. The method of claim 3 wherein said surface is a solid surface.

15. The method of claim 3 wherein said surface is a cell surface.

16. The method of claim 3 wherein said surface is a paramagnetic bead.

17. The method of claim 3 wherein said first and said second agent are covalently attached to said surface.

18. The method of claim 3 wherein said first and said second agent are noncovalently attached to said surface.

19. The method of claim 3 wherein said first and said second agent are indirectly attached to said surface.

20. The method of claim 3 wherein the cancer is selected from the group consisting of NHL, CLL, and CML.
